(19) United States
(12) Patent Application Publication
(10) Pub. No.: US 2011/0190815 A1
(43) Pub. Date: Aug. 4, 2011

(54) KNOTLESS SUTURE ANCHORS

(76) Inventor: Justin D. Saliman, Los Angeles,, CA (US)

(21) Appl. No.: 13/062,664

(22) PCT Filed: Sep. 8, 2009
(86) PCT No.: PCT/US09/56152

§ 371 Date: Apr. 19, 2011

Related U.S. Application Data
(60) Provisional application No. 61/095,261, filed on Sep. 8, 2008.
(60) Provisional application No. 61/103,197, filed on Oct. 6, 2008.
(60) Provisional application No. 61/106,896, filed on Oct. 20, 2008.

(51) Int. Cl. A61B 17/04

(52) U.S. Cl. 606/232

(57) ABSTRACT

Described herein are knotless suture anchors, and systems and methods for using knotless suture anchors. In general, a knotless suture anchor includes an anchor body configured to be anchored or embedded into bone, and a loop that extends from the anchor body as well as a loop-puller string that also extends from the anchor body. The loop can be contracted by pulling on the loop-puller string extending from the anchor. The anchor device is configured so that the loop can only be retracted into the anchor body, but not protracted or expanded. A suture (which may also be attached to the suture anchor) may be passed through the loop before it is contracted. The loop may be contracted so that it is drawn into the anchor body. Thereafter, the suture may be cut or trimmed.

### CROSS-REFERENCE TO RELATED APPLICATIONS

This application claims priority to the following U.S. Provisional patent applications: U.S. Ser. No. 61/095,261, titled “KNOTLESS SUTURE ANCHORS” (filed Sep. 8, 2008); U.S. Ser. No. 61/103,197, titled “KNOTLESS SUTURE ANCHORS” (filed Oct. 6, 2008); and U.S. Ser. No. 61/106,896, titled “KNOTLESS SUTURE ANCHORS” (filed Oct. 20, 2008).

### INCORPORATION BY REFERENCE

All publications and patent applications mentioned in this specification are herein incorporated by reference in their entirety as if each individual publication or patent application was specifically and individually indicated to be incorporated by reference.

### BACKGROUND OF THE INVENTION

The present invention relates to tissue repair. More particularly, the devices, systems and methods described herein relate to anchoring of sutures applied to biological tissue, such as tendons or ligaments, and/or biological implants to a bone.

Soft tissues, such as tendons and ligaments, generally are attached to bone by small collagenous fibers. These connections are strong and allow the tendons and ligaments to be flexible. When a tissue is torn away from the bone and requires repair, a surgeon may repair the detached soft tissue with sutures which are passed through bone tunnels and tied, or the sutures may be anchored to the bone using one or more suture anchors.

A variety of devices are available for attaching objects to bone, including screws, staples, cement, suture anchors, and sutures alone. These devices have been used to attach soft tissue, such as ligaments, tendons, muscles, as well as objects such as protheses, to bone. A suture anchor is a device which utilizes small anchors with suture materials attached thereto. A device, such as a screw, is inserted into the bone mass and anchored in place. After insertion of the anchor, the attached suture is passed through the tissue to be repaired. The tying of a knot in the suture is then required to secure the tissue to the bone. The process of passing the anchored suture through the soft tissue and tying a knot is time consuming and difficult to undertake in the tight space encountered during endoscopic surgery and sometimes even in conventional open surgery.

Unfortunately, tying sutures using suture anchors typically result in knots or fasteners (e.g., a portion of the suture anchor) which may remain in tissue, and can rub against the tissue, particularly joint tissues.

Although knotless suture anchors are know (see, e.g., U.S. Pat. No. 5,709,708), such suture anchors are often difficult to use and often require multiple complex steps for placement and for reduction of the involved tissues down to the bone anchor. Moreover, such devices may be impractical for use in small or narrow body regions such as joints (e.g., shoulder joints) in which there is little room for a surgeon to maneuver, and particularly to pass a suture. Large tied knots in spaces such as the glenohumeral joint of the shoulder may additionally cause damage to the surrounding cartilaginous surfaces secondary to mechanical abrasion, as the currently utilized suture material is infrequently bioabsorbable.

In addition, many of the known suture anchors are overly complicated to use, and require loading of the suture to be anchored before the device is inserted or anchored into the patient. This reduces the flexibility and usefulness of the procedures that may be performed with such devices. For example, the Vesalock is a knotless anchor provided by Johnson and Johnson in which sutures are fed through a loop, but the suture must be fed through the loop before the anchor is implanted. Arthrocare (see e.g., http://www.arthrocaresportsmedicine.com/files/datasheets/A1048A.pdf) offers a similar suture anchor, as do Arthrex (e.g., http://www.deviceinnovation.com/images/1172462330/11.jpg) and Tornier (e.g., http://www.fda.gov/cdrh/pdf8/K080335.pdf), however all of these devices require that the sutures to be anchored are pre-attached to the anchor prior to use.

Described herein are knotless suture anchors, systems including knotless suture anchors and methods of using them that may address these difficulties.

### SUMMARY OF THE INVENTION

Described herein are devices, systems and methods for knotless suture anchors. In general, a knotless suture anchor includes an anchor body configured to be anchored or embedded into bone, and a loop that extends from the anchor body as well as loop-puller string that also extends from the anchor body. The loop is formed of a string, which can be continuous with, or connected to, the loop-puller string. The loop extends from the anchor body, and can have a large opening. The loop can be a lasso. The loop can be drawn into the anchor body by pulling on the loop-puller string extending from the anchor. In some variations the loop is contracted by pulling on the loop puller. The loop puller may draw one end of the string forming the loop into the anchor body, or it may draw both ends of the loop into the body. The anchor device is configured so that the loop can only be retracted, but not expanded or protracted. A suture (which may also be attached to or within the anchor body) may be passed through the loop before the loop is contracted. The loop may be contracted so that it is drawn completely into the anchor body. Thereafter, the suture end and the loop-puller string may be cut or trimmed.

For example, described herein are knotless suture anchors comprising an anchor body configured to anchor into bone, a suture extending from the anchor body, a loop extending from the anchor body, wherein the loop is formed of a string, a loop-puller string connected or continuous with the loop so that pulling the loop-puller string reduces the length of the loop extending from the anchor body, and a one-way lock within the anchor body, wherein the loop-puller string or loop passes through the one-way lock, further wherein the one-way lock is configured to permit the loop-puller string or loop to be drawn through the one-way lock in only one direction.

In some variations, the anchor body comprises a conical distal end, or is otherwise shaped or adapted for insertion into the bone. For example, the anchor body may be adapted to grip the bone, so that it remains anchored into the bone permanently of for an extended period. Thus, the outer surface of the anchor body may be configured to anchor into bone. For example, the outer surface of the anchor body may be threaded.

The loop may also be referred to as a “suture loop.” The loop-puller string may be formed of any appropriately elongated, flexible material. For example, the string may be formed of a filament, thread, suture, strand, etc. The string may be made of a filamentous material, or a mono-filament. The string may be formed of natural or synthetic materials, including polymers, alloys, metals, and the like. The string may also be formed of any appropriate suture material. As described herein, the loop-puller may also be referred to as a suture end, or a “second suture end”, even though the loop-puller string may be made of non-suture materials.

Since the suture anchors described herein may be inserted for long-term use in a patient, the suture anchor may also include an additional means for securing a suture. For example, the suture anchor may also be used in conjunction with a cement, an adhesive, or the like, to help secure the suture in place, once a suture has been passed through the loop and the loop has been constricted around it and retracted into the anchor body. For example, a locking bead may be included in the anchor body. In some variations a locking bead (which may be a rounded bead, or may have irregularly shaped sides) may be threaded on the string forming the loop so that constricting the loop may cause the locking bead to engage the suture and/or the loop within the anchor body. In some variations, activatable cement may be included within the anchor body, wherein the activatable cement is configured to activate after reduction in the length of the loop. For example, constricting the loop may rupture a container of activatable cement within the anchor body. In some variations the locking bead includes an activatable cement.

The knotless suture anchors described herein may also include a suture shuttle on the distal end of a suture that is included as part of the knotless suture or a system including a knotless suture. The suture shuttle may be for use with a continuous suture passer, such as the continuous suture passers described in U.S. patent application Ser. No. 11/773,388, titled (“METHODS AND DEVICES FOR CONTINUOUS SUTURE PASSING”) filed on Jul. 3, 2007.

In some variations, the loop is connected to a suture extending from the suture anchor and the loop-puller string. In other variations it may be more effective for a suture end extending from the knotless suture anchor to be secured within the suture shuttle (e.g., in the shuttle body and not be connected to the loop. For example, the suture end may be tied, tethered, glued, affixed, or otherwise secured within the suture anchor. In some variations the suture anchor does not include a suture.

The loop is generally formed of a string, as mentioned. The loop may be a fixed loop (e.g., having a fixed open shape/size), or it may be constrictable so that the size of the loop can be changed. The loop may be a lariat or lasso. The loop may be opened (e.g., having ends that are not directly connected and are located within the anchor body. For example, one end of the loop may be secured within the anchor body, while the other end of the loop is connected (or continuous) with the loop-puller string extending from the knotless suture anchor. In some variations the string forming the loop is not attached to the suture anchor body, but is attached to the loop-puller string, or is formed of the same string forming the loop-puller string. This string may pass through the anchor body, but is not fastened to the anchor body, although the passage of the string through the anchor body is limited by the one-way lock.

Any appropriate one-way lock may be used, as described herein. For example, the one-way lock may include a hinged member such as a hinged pin or plane that allows the loop and/or loop-puller string to be pulled distally (away from the suture anchor), causing the constriction of the loop outside of the anchor body. In some variations the lock is engaged by one or more members attached to the string forming the loop and/or loop-puller string. For example, the lock may include one or more locking clips or beads secured to the string forming the loop. These locking clips or beads may be passed through a portion of the one-way lock (e.g., a channel, tunnel, passage, etc.) within the anchor body in one direction, but not in the reverse direction. A cam system may alternatively or additionally be employed to enable a one-way locking mechanism.

As mentioned, although the loop may be called a “suture loop”, it is to be understood that any of the loop or the loop-puller string may be made of any appropriate flexible material, including traditional and non-traditional suture materials. Other string materials or fabrics (including metals, alloys, polymers, plastics, rubbers, etc.) may be used.

Also described herein are knotless suture anchor comprising a bone-penetrating anchor body, a first suture end extending from the anchor body, a loop extending from the anchor body, a one-way lock within the anchor body configured to permit the loop to contract but not expand, and loop-puller string extending from the anchor body wherein pulling the loop-puller string contracts the loop.

Also described herein are knotless suture anchors comprising: an anchor body configured to be received in bone; a loop extending from the anchor body, wherein the loop is formed of a string; a one-way lock within the anchor body configured to permit the loop to contract but not expand; and a loop-puller string extending from the anchor body configured so that pulling the loop-puller string contracts the loop. Any of the variations elements described above may be used in these variations as well.

Also described herein are knotless suture anchor comprising: an anchor body, a string forming a loop that extends from the anchor body, wherein distal end of the string also extends from the anchor body, and a one-way lock within the anchor body, wherein the string passes thorough the anchor body, and further wherein the one-way lock is configured to permit the loop to contract but not expand. Any of the variations elements described above may be used in these variations as well.

Any of the knotless suture anchors described herein may also be part of a system, such as a knotless suture anchor delivery system. For example, a system may comprise: a knotless suture anchor, a suture, and an elongate sheath configured to releasably secure the knotless suture anchor at its distal end, wherein the elongate sheath comprises an inner lumen in which the suture and the loop-puller string may extend. The knotless suture anchor may include: an anchor body, a loop extending from the anchor body, a one-way lock within the anchor body configured to permit the loop to contract but not expand, and a loop-puller string extending from the anchor body configured so that pulling the loop-puller string contracts the loop.

Methods of using a knotless suture anchor to anchor one or more sutures are also described. For example, described herein are methods of securing a material to a bone with a knotless suture anchor including the steps of placing a knotless suture anchor in bone (wherein the knotless suture anchor comprises an anchor body, from which a loop and a loop-puller string extend, and wherein the knotless suture anchor also comprises a one-way lock within the anchor body configured to permit the loop to contract but not expand); passing a suture through or around a target, and then pushing the suture through the loop while the loop is external to the knotless suture anchor body; pulling the loop-puller string to contract the loop into the anchor body; and cutting the loop-puller string and the suture.

In some variations, the methods may also include pushing one or more sutures through the loop before drawing the loop into the anchor body.

The step of passing the first suture end through or around a target may comprise passing the first suture end through or around a soft tissue. Further, the step of passing the first suture end through or around a target may comprise passing the first suture end through or around an implant material. The method may also include activating a cement within the anchor body to secure the loop and suture end.

### BRIEF DESCRIPTION OF THE DRAWINGS

FIG. 1A shows one variations of a knotless suture anchor as described herein.

FIG. 1B shows the knotless suture anchor of FIG. 1A in the anchored configuration.

FIGS. 2A-2C show variations of the anchor bodies for knotless suture anchors.

FIGS. 3A and 3B show cross-sections through one variation of a knotless suture anchor.

FIG. 4 is a cross-section through one variation of a knotless suture anchor

FIG. 5 is a cross-section through another variation of a knotless suture anchor.

FIG. 6 is a cross-section through another variation of a knotless suture anchor.

FIG. 7A-7D illustrate implantation of the knotless tissue anchor in bone.

FIG. 8A-8G illustrate a method of suturing and anchoring using one variation of the knotless suture anchor described herein.

FIG. 9A shows another variation of a knotless suture anchor, as described herein.

FIGS. 9B and 9C illustrate operation of the knotless suture anchor shown in FIG. 9A.

FIGS. 10A through 10C illustrate operation of a knotless tissue anchor to anchor two sutures.

FIGS. 11A and 11B illustrate operation of another variation of a suture anchor.

FIG. 12A shows another variation of a knotless suture anchor having a fixed loop which may be drawn into the anchor body to anchor a suture, as illustrated in FIG. 12B-12C.

FIG. 13 illustrates a knotless suture anchor, such as the one shown in FIG. 12A or 11A, securing a region of tissue.

FIG. 14A-14E illustrates use of a knotless suture anchor.

FIG. 15A-15C is another example illustrating the use of a knotless suture anchor.

### DETAILED DESCRIPTION OF THE INVENTION

In general, a knotless suture anchor includes a bone anchoring body region (e.g., an anchor body), a loop that is open and extends from the body, and a loop-puller string extending from the body that can be pulled to constrict the loop and/or draw it into the anchor body. The anchor body includes a one-way lock that prevents the loop from opening (e.g., allowing it to be contracted by pulling the suture end, but preventing the suture end moving in the opposite direction). A suture may also extend from the anchor body, and this suture can be passed through or around tissue and anchored to the suture anchor.

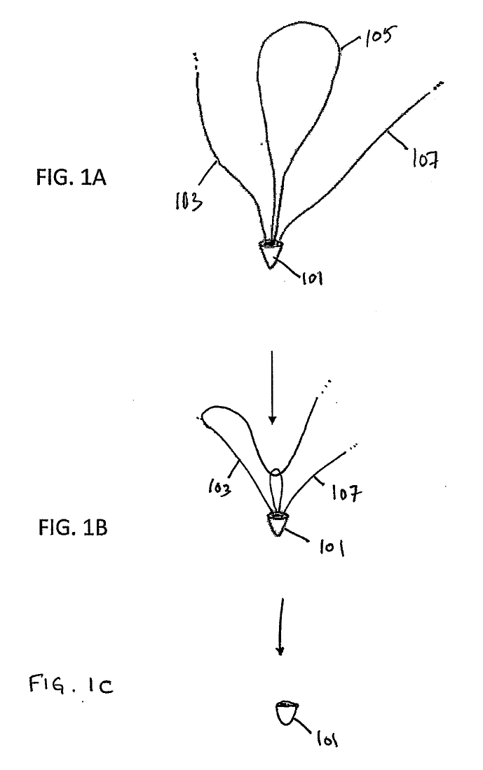

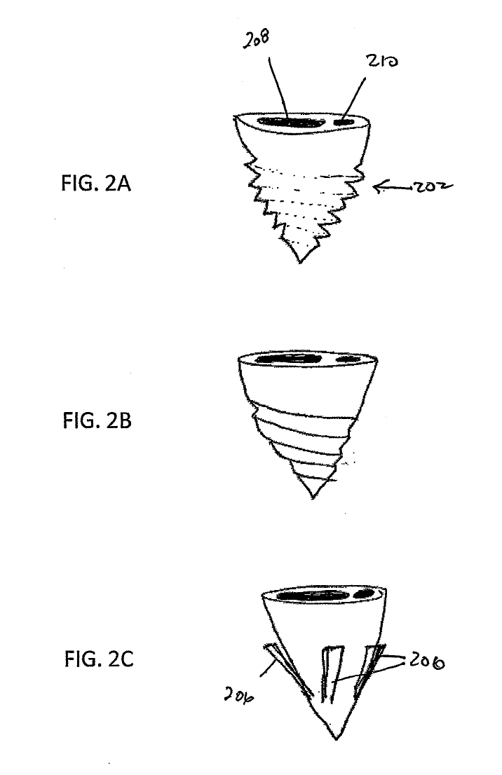

FIG. 1A shows one variation of a knotless suture as described. In FIG. 1A, the anchor body 101 is conical or tapered, having a more pointed end. In practice, any anchor body shape may be used, particularly shapes that are adapted for insertion or implantation into bone. Examples of anchor body regions are described below with reference to FIGS. 2A-2C. In the variation shown in FIG. 1A, a loop 105 extends from the anchor body, as do a suture 103 and a loop-puller string 107. The figures are not shown to scale. In operation, the suture end 103 is affixed at one end (e.g., the distal end) within the anchor body, and extends from the anchor body. The loop 105 also extends from the anchor body 101, and may be formed from a similar suture material as the suture 103. One end of the loop 105 is typically connected to an end (e.g., the distal end) of the loop-puller string 107. The loop-puller string 107 may be continuous with the loop. The loop-puller string 107 also extends from the anchor body 101. As will be described in greater detail below, the loop-puller string and the suture anchor are engaged by a one-way lock (not visible in FIG. 1A) within the anchor body. This one-way lock prevents the loop-puller string 107 from being drawn back into the anchor body 101, but allows the loop-puller string 107 to be pulled out of the anchor body 101, thereby collapsing the loop 105, and drawing the loop into the anchor body 101. The loop-puller string may be marked (e.g., colored, etc.) or may be attached to a marker that helps differentiate it from other sutures such as the suture 103. Operation of the tissue anchor is illustrated in FIG. 1B.

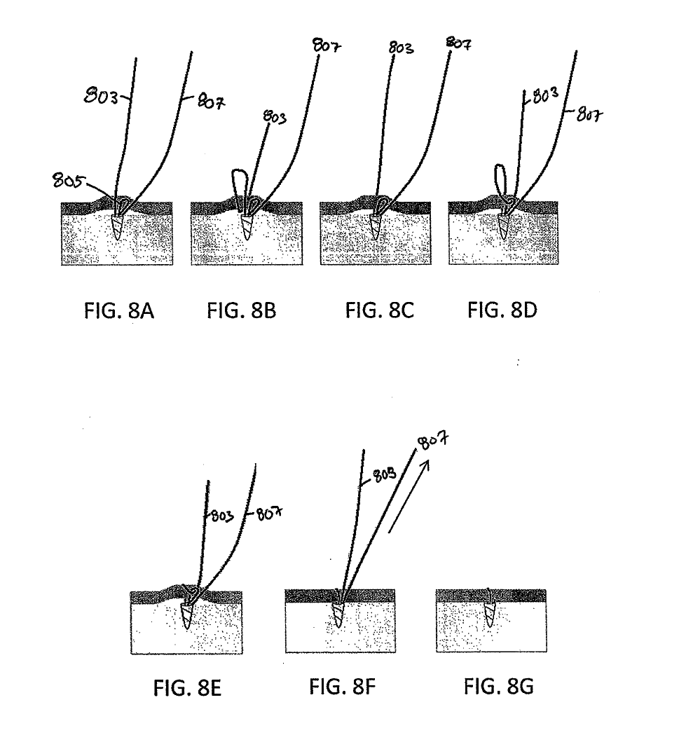

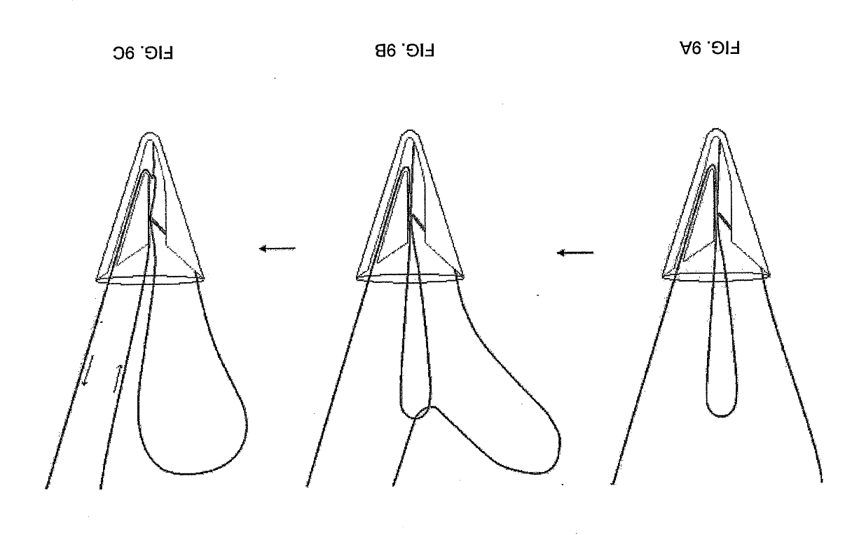

In FIG. 1B, the loop 105 has been contracted by pulling on loop-puller string 107 until the loop 105 has been completely collapsed, and drawn into the anchor body 101. As will be described in greater detail below, another suture or sutures, or a portion of an implant, may be passed through the loop 105. FIG. 1B shows the proximal end of the suture end 103 has been passed through the loop. In some variations, an entirely different suture may or material may be passed through the loop. The loop may be “collapsed” by pulling on the loop-puller string 107, drawing the loop into the anchor body, and therefore anchor the suture or material passed through the loop to the anchor body, and thus the bone or other material to which the anchor body is attached. It may be beneficial to draw the loop 105 completely into the anchor body 101 both to secure the knotless suture anchoring of the suture or other material, and also to prevent the anchored material from being exposed to the surrounding tissue. This may also help ensure that the tissue or other material being secured is reduced and secured to the bone (or tissue or other material that it is being secured to) to prevent gap formation at the repair site. Anchoring the material within the anchor body may protect the surrounding tissue from rubbing against the suture material. Once the loop 105 has been drawn into the anchor body, the loop-puller string extending from the anchor body may be cut, as shown in FIG. 1C and (as described below) in FIG. 8G.

The anchor body 101 region may therefore be configured so that it can be secured to a region of the body, and particularly bony regions, while presenting a smooth or atraumatic surface to the surrounding tissue, once anchored in the body. In addition, the anchor body may be configured so that the anchor body can be readily secured within the patient, including bone regions. FIGS. 2A-2C illustrate variations of anchor bodies that are adapted for anchoring in bone.

For example, FIG. 2A is one variation of an anchor body having ridged or notched regions 202 along the outside of a tapered region of the body. This tapered region may be configured for insertion into bone, and the ridges/notches may help secure the anchor in place. FIG. 2B shows another variation, in which the anchor body is tapered and threaded, which may also help insertion into bone, by allowing the device to be screwed into a bone. In some variations, the anchor body is adapted to secure the device by including one or more expanding anchor regions. For example, the variation shown in FIG. 2C includes arms 206 that can flange out from the anchor body, preventing the anchor from being readily withdrawn, once it has been implanted into a bone. The anchor body may also include one or more passages for bone cement and/or additional bone screws or pins. Thus, the anchor body may be secured firmly to bone. In some variations, the anchor body may be configured to be releasably secured to bone. For example, the anchor body may be resorbsorbable or bioabsorbable. In some variations, the anchor may promote bone in-growth, and may include openings or regions for bone in-growth. The anchor body may also include bone growth promoting materials.

The region of the anchor body that is not configured to be embedded into the bone may be adapted so that it presents an atraumatic surface. For example, this surface may be smooth, or may be compliant (e.g., coated or fashioned of a compliant material), and/or lubricious. In addition, this surface may also include one or more openings from which the loop and suture end(s) extend. In FIGS. 2A-2C the anchor body includes two openings 208 and 210. For example, the loop and a suture end may extend from the first opening 208, and the loop-puller string, which may be drawn to collapse the loop, may extend from the second opening 210. Although one of the openings 208 shown in FIGS. 2A-2C is larger than the second opening 210, in some variations, the openings may be the same size. In some variations only a single opening from which the loop and the suture end(s) and loop-puller string extend is used. In other variations, three or more openings are included. Although the variations illustrated include one suture extending from the anchor body, in some variations, multiple sutures may be connected and extend from the anchor body.

The anchor body region may be formed of any appropriate material(s), including metals, alloys, ceramics, plastics, rubbers, polymers, biologics or some combination thereof. Similarly, the material forming the loop and the loop-puller string and any suture ends extending from the anchor body may be formed of a suture material, or any other appropriate material. In general, the material forming the loop, loop-puller string, and suture are formed from a flexible material. This material may be absorbable or biodegradable. In some variations, the material is formed of a traditional suture material (e.g., surgical gut, chromic suture materials, polylactic acid, capraolactone, polyglycolic acid, nylon, polypropylene, polyester, silks, etc.), and may be monofilament, woven, braided, or the like. The material forming the loop and/or suture end(s) may also be formed of flexible metals, alloys, polymers, or the like. Any appropriate size (length and/or diameter) may be used.

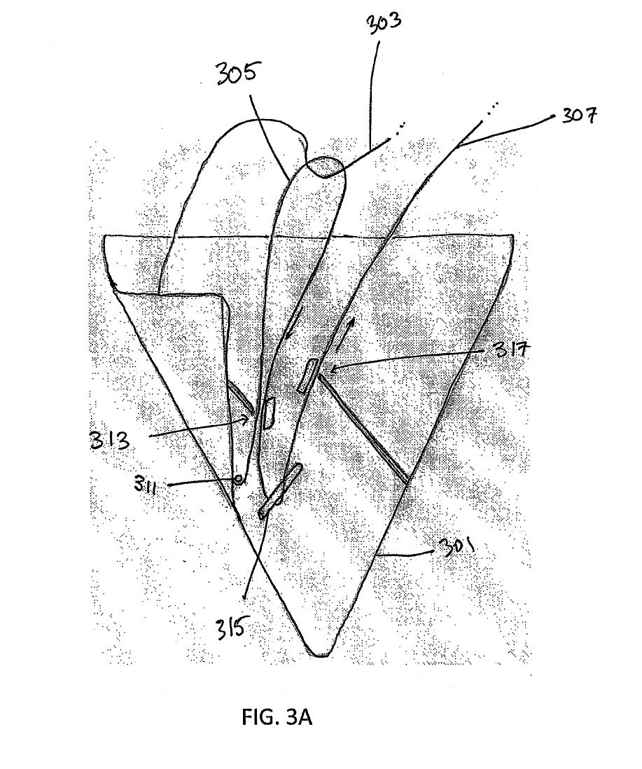

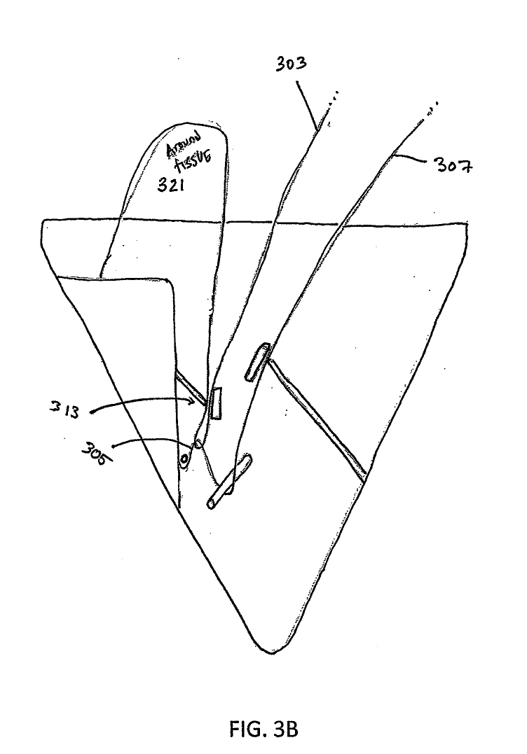

As mentioned above, the anchor body typically includes a one-way lock. FIGS. 3A-3B illustrate a partial cross-section through an anchor body schematically illustrating the operation of one variation of a knotless suture anchor. In FIG. 3A, the loop 305 extends from the anchor body 301. One end 311 of the loop 305 is continuous with the suture end 303 extending from the anchor body, and the other end of the loop is continuous with the loop-puller string 307 extending from the anchor body 301. The material forming the loop and the suture and loop-puller string passes around a first pulley 311, through a first one-way lock 313, forms a loop 305, and then passes around a second pulley 315 and through a second one-way lock 317. The pulley around which the material passes may be a rolling pulley, or simply a pin, bar, or the like.

In FIG. 3A, the one-way locks are configured to sandwich the string forming the loop (and suture and loop-puller string) between two or more surfaces in such a way that the string/suture can only move freely in one direction through the one-way locks, preventing the loop from loosening. Thus, these two locks are oriented in opposite directions in this variation. For example, the first lock 313 allows the suture 303 to be pulled, thereby contracting the loop. The second lock 317 likewise allows the loop-puller string 307 to be pulled to contract the loop, but prevents the loop from loosening. As the ends of the suture and loop-puller string are pulled, the loop may pull into the anchor body 301, as shown in FIG. 3B.

In FIG. 3B, the interface between the suture end 303 and the loop 305 has been withdrawn completely into the anchor body, and has passed through the first one-way lock 313, effectively locking the suture 303 so that it cannot loosen as well. Before the suture was passed through the loop 305, it was first passed through a tissue or device to be anchored 321. Once anchoring is complete, the loop-puller string end 307 may be trimmed or cut, as may the suture end 303. In some variations the anchor body may also include a bobbin, cam, or spring that will pull the cut end of the suture and/or loop-puller string extending from the anchor body into the anchor body once it has been cut. For example, putting tension on the loop-puller string may extend the spring or bobbin and the loop-puller string end may be cut while the tension is maintained. After cutting and release of the tension, the spring or bobbin may then relax back down, drawing the cut end into the anchor body. Typically this spring or bobbin is located in the anchor body after any one-way lock.

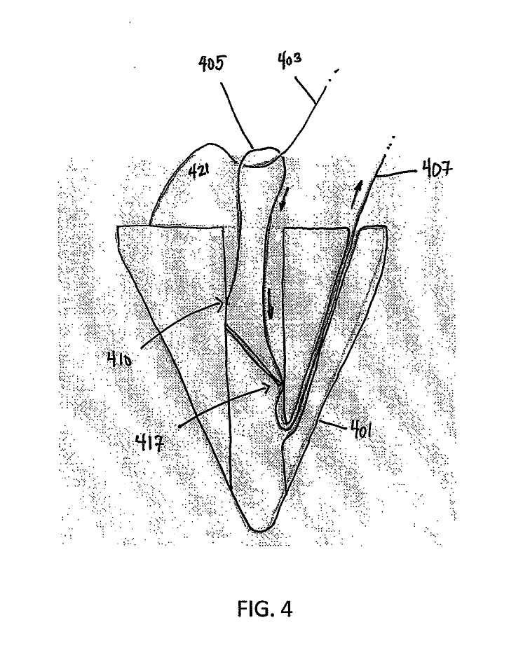

In some variations, one end of the loop is secured to the inside of the anchor body, and the distal end of the suture extending from the anchor body may also be attached to the inside of the anchor body. FIG. 4 illustrates an example of such a variation. In FIG. 4, the distal end of the first suture end extending from the anchor body is secured within the anchor body, and the distal end 410 of the material forming the loop 405 is secured to a wall of the inside of the anchor body 401. The other end of the material forming the loop 405 is continuous with the material forming the loop-puller string 407 extending from the anchor body 401. The loop passes through a one-way lock 417 before exiting anchor body 401 as the loop-puller string 407. The suture 403 passes through or around a tissue (or implant 421) and then through the loop 405. Pulling on the loop-puller string extending from the anchor body 407 will constrict the loop, eventually drawing the loop 405 and a portion of the suture 403 into the anchor body, where it may again be pulled below the one-way lock 417, securing it in the anchor body 401.

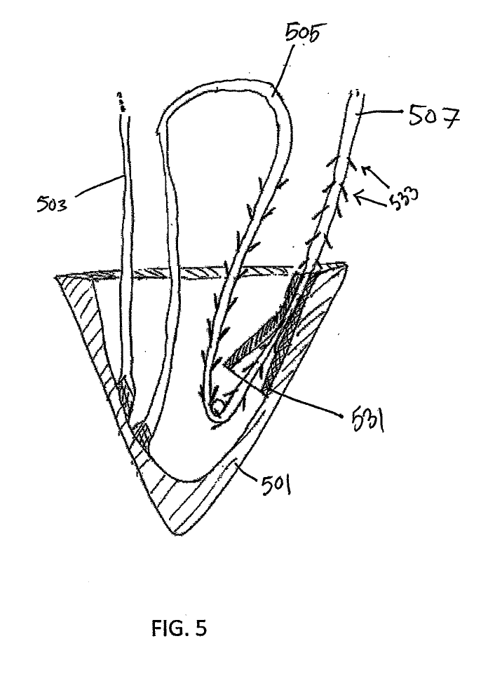

FIG. 5 shows another variation of a knotless suture anchor, including a different variation of the one-way anchor valve that prevents the material forming the loop 505 and the loop-puller string extending 507 from anchor body 501 from moving in more than one direction. In this example the one-way valve includes a funnel-like opening 531 into which the string of the loop-puller string and/or loop passes. The string (e.g., suture) material forming a portion of the loop 505 and the loop-puller string 507 in this example includes a plurality of locking clips 533. These locking clips may be arms or sheets extending from the suture that will compress when drawn through the funnel 531 of the one-way lock towards the narrow region, then expand after exiting, preventing the string from moving back through the lock. In some variations these strings forming the loop and/or loop-puller string include beads, knots, or other structures which may be rigid or semi-rigid, and the funnel portion of the one-way lock is slightly flexible so that it can expand as the string is drawn in one direction; the configuration prevents the string from being pulled in the opposite direction.

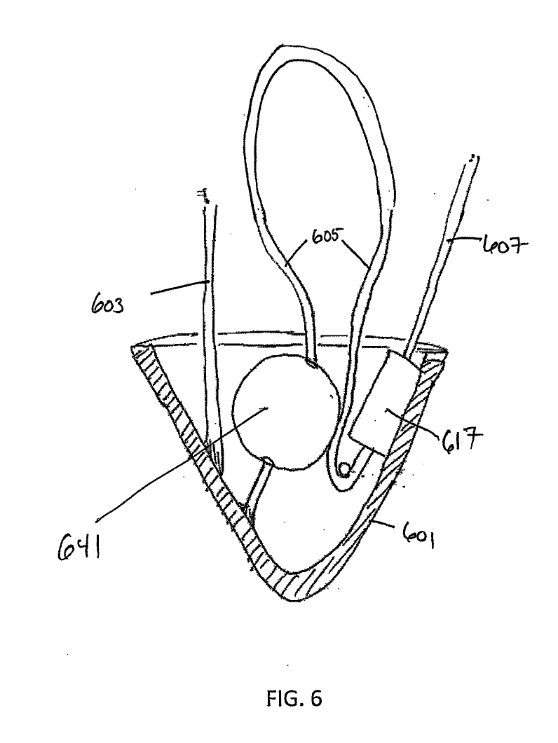

In some variations an additional locking mechanism is included for securing a material (e.g., the suture) captured by the loop within the anchor body. For example, a locking mechanism may include a locking bead as illustrated in FIG. 6. In FIG. 6, the locking bead 641 is threaded on the distal end of the loop 605. After a suture (e.g., the first suture end 603) is passed through the loop 605, and drawn into the anchor body 601 with the loop 605, it may be held secured against the inner surfaces of the anchor body 601 as the loop is secured by the one-way lock 617. Although a spherical bead is shown in FIG. 6, other shapes may be used, including shapes with one or more edges or points.

In addition or instead of locking beads, one or more materials may also be used to help secure the suture(s) and loop within the anchor body. For example, cement may be included. In some variations the cement is activatable or releasable, so that it becomes active only after the loop and/or suture has been secured within the anchor body. In some variations a crushable or frangible packet or container of cement is released after the loop has been drawn into the anchor body. In some variations, the cement may be activated by bringing two or more materials together. For example, the distal region of the first suture end may be treated with a material that bonds to the distal end of the loop; these two regions typically only meet once the loop has been withdrawn into the anchor body. In one variation the locking bead described in FIG. 6 is frangible or includes frangible regions that release a cement material after the loop has been drawn into the anchor body. Any appropriate cement may be used, particularly biocompatible cements (e.g., polymethylmethacrolate, cyanoacrylates, etc.).

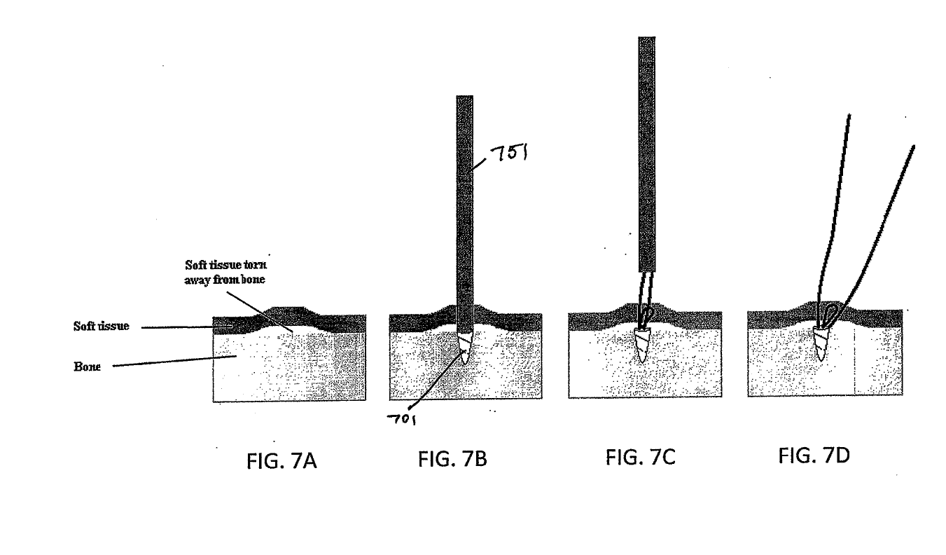

In operation, the knotless suture anchors described herein may be used by first anchoring to a bone or other body region to which the tissue and/or implant is to be secured. In some variations the knotless tissue anchor may be secured to an applicator which may assist in placing the anchor in the bone or other tissue. FIG. 7A-7C illustrates the use of an applicator for placing one variation of a knotless tissue anchor. In this variation the knotless tissue anchor 701 is secured to the distal end of an applicator including a sheath 751. The sheath is an elongate member having an inner lumen through which the loop, loop-puller string, and any suture attached to the device, extend. The sheath may be relatively stiff, so that it can be used to apply force to insert the device into the bone. FIG. 7A shows a bone region and a region of soft tissue that has been pulled away from the bone. The methods described herein may be used to re-attach the soft tissue to the bone. For example, in FIG. 7B, a knotless suture anchor is being secured into the bone, using an applicator. In some variations the bone may be prepared before this, for example, by drilling a hole or opening into which the body of the knotless suture anchor will be secured. In other variations, the body of the knotless suture anchor may itself be adapted for securing into the bone. In other variations, bone cement, bone screw, or other mechanism for fixing the knotless suture anchor in the bone may be used.

The knotless suture anchor may be released from the sheath of the applicator after it has been inserted, as shown in FIG. 7C. The applicator may be withdrawn, leaving the loop, loop-puller string, and suture end exposed, as shown in FIG. 7D.

FIGS. 8A-8G illustrate the use of a knotless suture anchor such as the one shown in FIGS. 7-7D after it has been secured to a tissue (e.g., bone). In FIG. 8A, the first suture end 803 and the loop-puller string 807, as well as the loop 805 are shown extending from the implanted anchor body. In FIG. 8B, the first suture end is then passed through or around the tissue to be secured by the knotless suture anchor. Any appropriate suturing technique or method may be used to suture the tissue with the first suture end. For example, the first suture end may include a shuttle or other attachment so that it can be used with a continuous suture passer. This may be particularly beneficial for passing the suture multiple times through the tissue, or passing it through the tissue and then through the loop, as required here. Continuous suture passers may be advantageous or even necessary when the tissue being secured is in hard-to-reach or hard-to-maneuver regions, such as the glenohumeral joint for labral repairs or the subacromial space of the shoulder for rotator cuff repairs, etc. In some variations the proximal end of the first suture end is adapted for use with a continuous suture passer.

In FIG. 8D the suture end is then drawn around (or back through) the tissue so that it can pass through the loop of the knotless tissue anchor. As mentioned above, any number of stitches through the tissue may be performed with the suture before passing through the loop of the suture anchor. In FIG. 8E the suture is secured by drawing it through the loop 805, and thereafter the loop is constricted by pulling on the loop-puller string 807, as shown in FIG. 8F. The loop-puller string may be drawn until the loop is completely retracted within the anchor body, as shown, securing the tissue and the suture 803 within the anchor body. In FIG. 8G the loose suture end 803 and loop-puller string 807 have been clipped or cut.

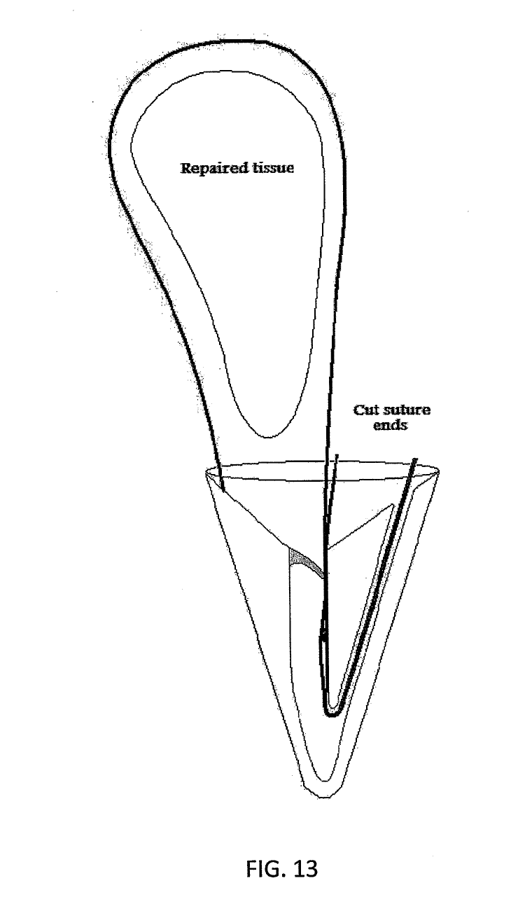

FIG. 9A illustrates another variation of a knotless suture anchor. In this variation the knotless suture anchor includes a suture attached within the anchor body, and a loop and loop-puller string extending from the anchor body. The suture may be passed through the loop and secured within the anchor body, as illustrated in FIGS. 9B and 9C. As the suture is drawn into the anchor body, it may loop around tissue (not shown, though see FIG. 13), anchoring the tissue in place as the suture is anchored. While drawing on the loop-puller string to lock the suture in place, the suture may be drawn to contract around the tissue (not shown). The suture may be anchored in the anchor body by pulling the loop until it is secured within the anchor body (e.g., so that the loop-puller string cannot be pulled any further).

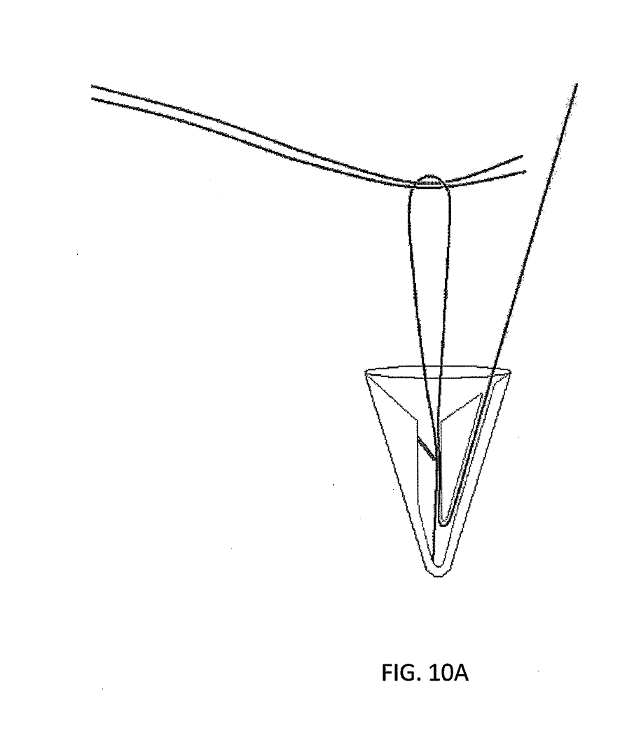

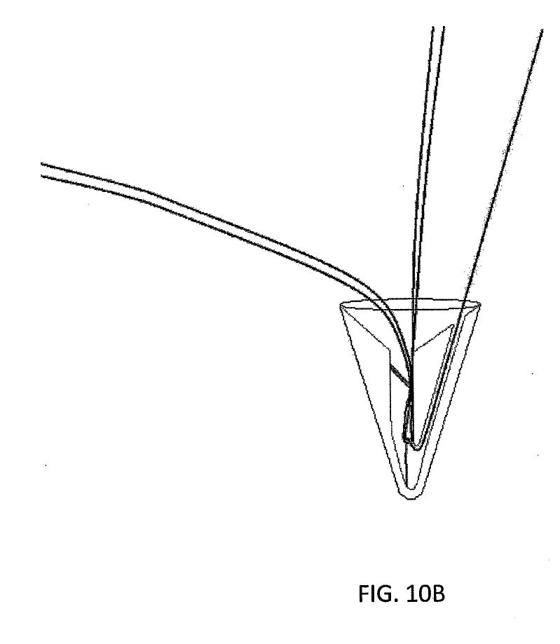

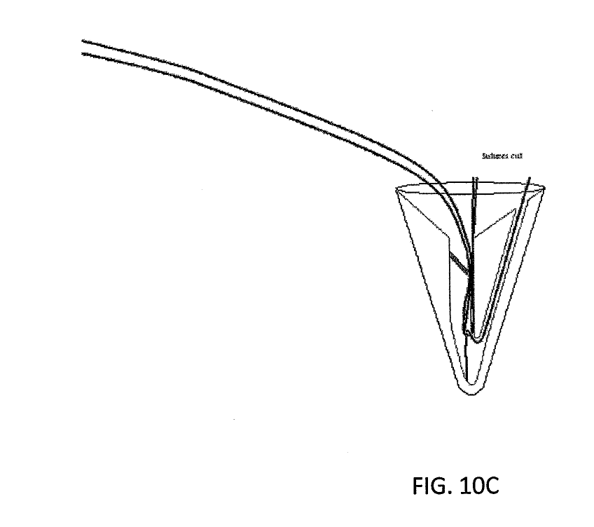

FIGS. 10A-10C illustrate another variation of a knotless suture anchor similar to those described above, but used to secure more than one suture. In the knotless suture anchor shown in FIG. 10A, a suture is not initially attached to the anchor body (e.g., compare to FIG. 9A). In this example, two sutures (drawn as parallel in this figure, although they may be oriented differently) are pushed or otherwise passed through the open loop extending from the suture body, and the loop is then drawn into the suture body to lock the plurality of sutures in position. FIGS. 10B and 10C illustrate this process. In FIG. 10C, one end of the sutures are cut.

Examples such as this may be beneficial when attempting to knotlessly anchor suture ends that have been passed through free tissue or passed from a different anchor through tissue (and then to an anchor). For example, this technique may be part of a “double row” of suturing used for rotator cuff repair.

In general, the sutures described herein may be pushed, pulled, shuttled, relayed or otherwise passed through the loop. Examples of suture passers that may push or shuttle the suture through the loop of the knotless suture anchor were previously incorporated by reference. Because the method using these devices may be performed deep within tissue, including as part of an arthroscopic or minimally invasive procedure, tissue passers such as those referred to may be particularly useful.

In all of these examples, the loop is external to the anchor body when the suture is passed through it. The loop may be relatively small, and difficult to pass a suture through. In operation, a suture may not need to be passed completely through the loop in order to be anchored. For example, a suture may be anchored by passing even a loop or section of an elongate suture through the loop, then contracting the loop into the anchor body with the portion of suture within the loop (e.g., a doubled-over region of suture), to anchor the suture in place. In many of the methods of operation of the knotless suture anchor described herein, the suture, whether initially anchored to the knotless anchor or from another location or anchor, is typically fed through the loop while the loop is external to the anchor body.

As mentioned, the loop and loop-puller string may be formed of any appropriate material(s), and may be part of the same string. For example, the string forming the loop and/or loop-puller string may be formed of suture, wire, cord, Nitinol, etc. For example, the string forming the loop-puller string and/or loop may be a coated cable.

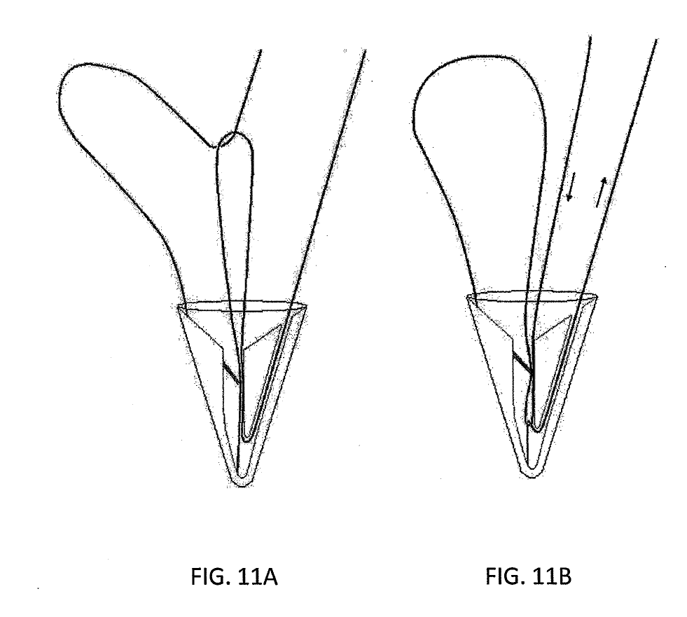

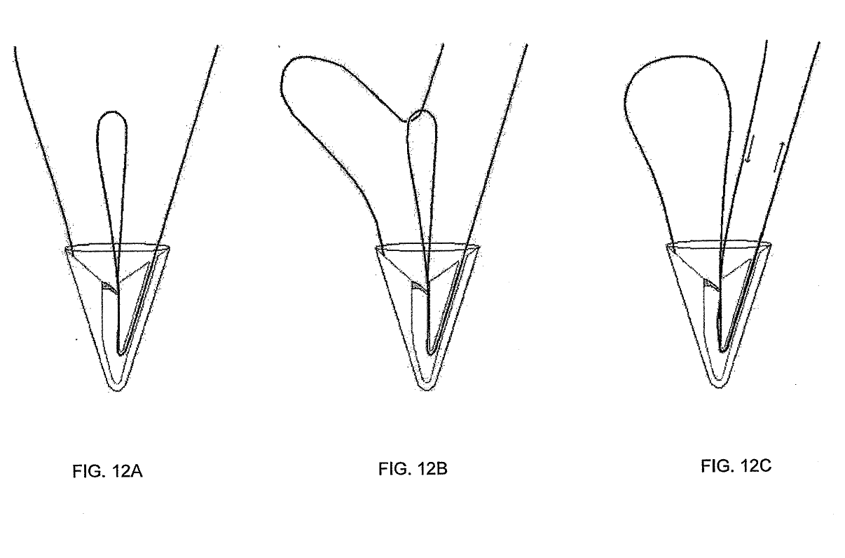

In some variations, the loop and/or loop-puller string are not directly attached to the anchor (e.g., anchor body). Instead, the loop is connected to the loop-puller string only, so that no end of the loop is attached to the anchor body. In this variation the loop may be a lasso-type configuration, in which one end of the string forming the loop attaches to the loop-puller string to form the loop from the loop-puller string. This is illustrated in FIGS. 11A-11B. In some variations the loop may be of a fixed size, since the loop is formed by tying, or affixing a free end a string to a doubled back region of the string. In other variations, the loop formed in the string may be expanded or contracted, lasso-like. In variations in which the loop has a fixed size, the loop rather than just the loop-puller string, may be drawn into the one-way lock, and held in position. For example, in FIG. 11B, drawing on the loop-puller string pulls the loop into the anchor body, and into the one-way lock in the anchor body. FIGS. 12A-12C illustrate another variation in which the loop extending from the anchor body is a fixed size, and the loop is drawn into the anchor body by pulling on the loop-puller string.

FIG. 13 illustrates a variation similar to that shown in FIG. 12A after anchoring a region of repaired tissue. In this example the suture (shown as being attached to the interior of the anchor body, has been passed around or through a region of tissue to be repaired, and passed through the loop, as illustrated in FIG. 12B, for example. The loop-puller string has been pulled to draw the loop into the anchor body. As the loop-puller string is drawn in, first the loop-puller string, and then the loop, is drawn into the one-way lock so that the loop cannot be extended from the anchor body again. Drawing the loop, with the suture captured in it, into the anchor body effectively locks the suture in the anchor, and secures the tissue in position, as shown.

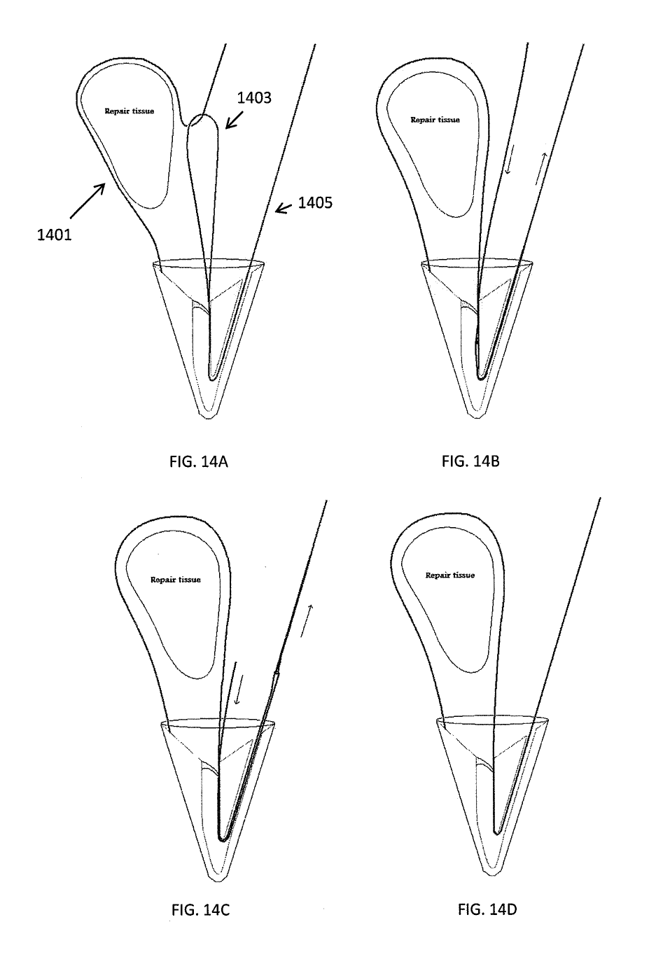

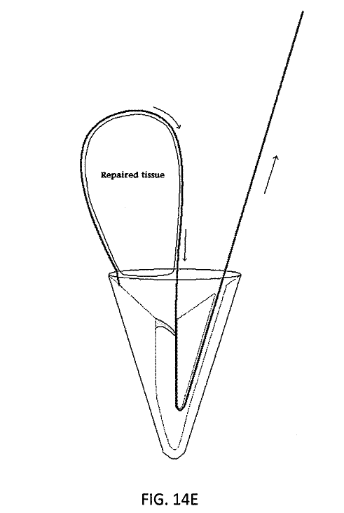

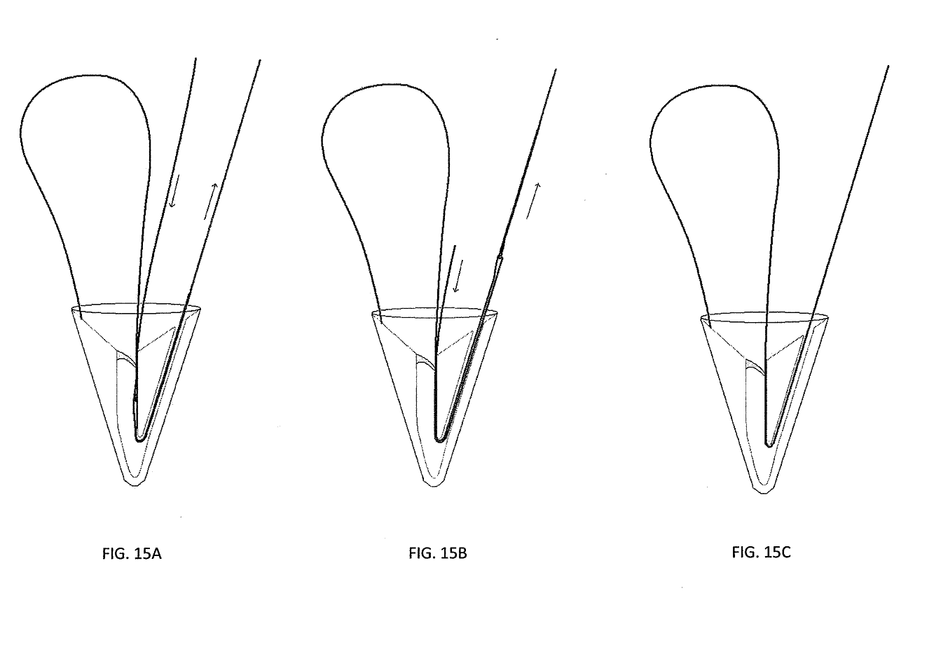

FIGS. 14A-14E illustrate the operation of another variation of a knotless suture anchor as described herein. In this example, the suture being anchored is pulled fully through the anchor so that the loop string is completely removed from the anchor and the patient. The suture can then be tightened/cinched down to pull the tissue down to the bone by simply pulling on the suture end. The suture engages the one-way lock within the anchor body, and is therefore held in place. Afterwards, the suture may be removed from the patient. FIG. 15A-15C shows a similar example. In FIG. 14A, the suture anchor includes an anchor body, from which a loop string 1403 and a loop-puller string 1405 extend. An optional suture 1401 is also shown extending from the body of the anchor. In operation, the suture anchor is first inserted and anchored into the body; the suture that will be used to pass through or around tissue 1401 is not passed through the loop 1403 until after the suture anchor has been placed and secured into the body (prior to the step shown in FIG. 14A). After passing a suture (such as the attached suture 1401, although a suture that has not been previously attached to the suture anchor may also be used) through or around the target tissue, the suture may be passed through the loop. For example, as described above, a suture passer may be used to pass the suture end through the loop. As illustrated in FIG. 14B, thereafter, the loop-puller string 1405 may be pulled to draw the loop (and therefore the portion of the suture passing through the loop) into the body of the knotless suture anchor. In the variation shown in FIG. 14A-14E, the loop is drawn completely out of the suture anchor, while continuing to pull the suture 1401 through the suture anchor, as shown in FIG. 14C. In this case, the suture is sufficiently long so that it can be pulled completely through the suture anchor and out of the patient by the loop. The suture is engaged by the one-way lock within the suture anchor, so that once it is pulled by the loop (e.g., in the direction shown by the arrows), it cannot be drawn in the opposite direction. In FIG. 14D, the suture 1401 has been completely passed through the suture anchor, and the distal end of the suture 1401 is grasped by a surgeon (not shown) either within or outside of the patient, so that the distal end can be further drawn into the suture anchor. Pulling the suture further into the suture anchor at this point may draw the anchored material (“repair tissue”) towards the anchor body, or at least tension the suture and thereby secure the tissue. This is further illustrated in FIG. 14E. Thereafter, the portion of the suture extending from the suture anchor which is not passing through or around the repair tissue may be trimmed or cut, as mentioned above. FIGS. 15A-15C illustrates a similar variation, without showing the tissue being repaired.

In any of the variations described herein, the suture to be anchored is not passed through the loop of the suture anchor until after the anchor is placed and/or anchored into the body. As mentioned above, the suture is passed into the loop once the anchor has been positioned. Thus, the suture is pulled, pushed, or otherwise passed through the loop after the anchor has been fully implanted within the body (e.g., bone).

Although the knotless suture anchors, systems and methods foregoing have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A knotless suture anchor comprising:
an anchor body configured to anchor into bone;
a suture extending from the anchor body;
a loop extending from the anchor body, wherein the loop is formed of a string;
a loop-puller string connected or continuous with the loop so that pulling the loop-puller string reduces the length of the loop extending from the anchor body; and
a one-way lock within the anchor body, wherein the loop-puller string or loop passes through the one-way lock, further wherein the one-way lock is configured to permit the loop-puller string or loop to be drawn through the one-way lock in only one direction.

2. The anchor of claim 1, wherein the anchor body comprises a conical distal end.

3. The anchor of claim 1, wherein the outer surface of the anchor body is configured to anchor into bone.

4. The anchor of claim 1, wherein the outer surface of the anchor body is threaded.

5. The anchor of claim 1, further comprising a locking bead threaded on the string forming the loop.

6. The anchor of claim 1, further comprising an activatable cement within the anchor body, wherein the activatable cement is configured to activate after reduction in the length of the loop.

7. The anchor of claim 1, further comprising a suture shuttle on the distal end of the suture.

8. The anchor of claim 1, wherein a portion of the suture is secured within the anchor body.

9. The anchor of claim 1, wherein one end of the string forming the loop is secured within the anchor body.

10. The anchor of claim 1, wherein the string forming the loop and the suture are connected.

11. The anchor of claim 1, wherein the one-way lock comprises a hinged member.

12. The anchor of claim 1, wherein the one-way lock comprises locking clips or beads secured to the string forming the loop.

13. The anchor of claim 1, wherein the loop is formed from the same string as the loop-puller string.

14. The anchor of claim 1, wherein the string forming the loop is selected from the group consisting of suture, wire, cord, or Nitinol.

15. A knotless suture anchor comprising:
an anchor body configured to be received in bone;
a loop extending from the anchor body, wherein the loop is formed of a string;
a one-way lock within the anchor body configured to permit the loop to retract into the anchor body, but not to protract; and
a loop-puller string extending from the anchor body configured so that pulling the loop-puller string retracts the loop.

16. A knotless suture anchor comprising:
an anchor body,
a string forming a loop that extends from the anchor body, wherein distal end of the string also extends from the anchor body; and
a one-way lock within the anchor body, wherein the string passes thorough the anchor body, and further wherein the one-way lock is configured to permit the loop to contract but not expand.

17. A knotless suture anchor delivery system, the system comprising:
a knotless suture anchor including:
an anchor body,
a loop extending from the anchor body,
a one-way lock within the anchor body configured to permit the loop to contract but not expand, and
a loop-puller string extending from the anchor body configured so that pulling the loop-puller string contracts the loop;
a suture; and
an elongate sheath configured to releasably secure the knotless suture anchor at its distal end, wherein the elongate sheath comprises an inner lumen in which the suture and the loop-puller string may extend.

18. A method of securing a material to a bone with a knotless suture anchor, the method comprising:
placing a knotless suture anchor in bone, wherein the knotless suture anchor comprises an anchor body, from which a loop and a loop-puller string extend, wherein the knotless suture anchor also comprises a one-way lock within the anchor body configured to permit the loop to retract into the anchor body, but not protract from the anchor body;
passing a suture through or around a target, and then pushing the suture through the loop while the loop is external to the knotless suture anchor body and after the knotless suture anchor has been secured in the bone; and
pulling the loop-puller string to contract the loop into the anchor body.

19. The method of claim 18, further comprising cutting the loop-puller string and the suture.

20. The method of claim 18, wherein the step of passing the first suture end through or around a target comprises passing the first suture end through or around a soft tissue.

21. The method of claim 18, wherein the step of passing the first suture end through or around a target comprises passing the first suture end through or around an implant material.

22. The method of claim 18, further comprising activating a cement within the anchor body to secure the loop and suture end.
